(19) United States
(12) Patent Application Publication
(10) Pub. No.: US 2017/0159089 A1
(43) Pub. Date: Jun. 8, 2017

(54) METHOD OF PRODUCING GLUCOSE FROM A CELLULOSIC BIOMASS USING ENZYMATIC HYDROLYSIS

(71) Applicant: Arbiom Inc., Durham, NC (US)

(72) Inventors: Lisette Tenlep, Big Stone Gap, VA (US); Pavani Mandali, Alpharetta, GA (US); Pratik Darvekar, College Station, TX (US); Laura Carter, Pound, VA (US); Cassidy Strange, Wise, VA (US); Rebekah Kendall, Roanoke, VA (US); Samuel Smith, Lebanon, VA (US)

(73) Assignee: Arbiom Inc., Durham, NC (US)

(21) Appl. No.: 15/368,649

(22) Filed: Dec. 4, 2016

Related U.S. Application Data
(60) Provisional application No. 62/263,593, filed on Dec. 4, 2015.

(51) Int. Cl. C12P 19/02; C12P 19/14

(52) U.S. Cl. CPC C12P 19/02; C12P 19/14

(57) ABSTRACT

The present invention relates to a method of producing glucose from a cellulosic biomass using enzymatic hydrolysis. The present invention also relates to a device for performing such method.

The present invention relates to a method of producing glucose from a cellulosic biomass using enzymatic hydrolysis. The present invention also relates to a device for performing such method.

The conversion of cellulosic polysaccharides into monosaccharides via enzymatic hydrolysis is widely recognized as the key process to enable feasible bioethanol production. In commercial processes high sugar concentrations are targeted, hence the initial suspended solids concentration in the hydrolyzate should preferably be at least above 20% (200 kg solids per ton of suspension). The main challenges associated with that are related to enzyme production and the low yields associated with high solids loadings that affect the process economy. Although high enzyme loadings typically result in an enhanced degree of conversion, their practically is determined by the cost of enzyme and the price of the targeted end products.

Different approaches have been followed, such as a conventional batch approach, wherein all ingredients are added initially into a single vessel, enzymatic hydrolysis is performed, and thereafter, the product(s) is (are) harvested. A refined version thereof is a fed batch process, wherein within the same reaction vessel in intervals, additional ingredients are added or replenished.

Although the conventional batch approach can operate at high solids content in enzymatic hydrolysis; the resulting mixture is difficult to mix and the enzyme loading cannot be lowered without impacting overall sugar yields. With the fed batch approach the solids can be elevated with less agitation impact in the enzymatic hydrolysis process, but lowering the enzyme loading without impacting the sugars concentrations and yields is not possible.

Accordingly, there is a need in the art to provide an improved process for enzymatic hydrolysis of a cellulosic biomass. According to a first aspect of the present invention, there is provided a method of producing glucose from a cellulosic biomass using enzymatic hydrolysis, wherein said enzymatic hydrolysis is performed in a multistage hydrolysis process in a plurality of hydrolysis vessels, said plurality of hydrolysis vessels being serially arranged such that there is a first hydrolysis vessel and, optionally, one or several hydrolysis vessels in between, wherein during said multistage process, each hydrolysis vessel contains a solid fraction SF which is biomass and a liquid fraction LF which is an aqueous solution, said multi-stage process having a front-end at said first hydrolysis vessel and a back-end at said last hydrolysis vessel, said multistage process involving an initial addition of enzyme to each hydrolysis vessel, said multistage hydrolysis process further involving in each hydrolysis stage the feeding of cellulosic biomass and enzyme at the front-end, the removal of enzymatically treated biomass at the back-end, the addition of water at the back-end, and the removal of aqueous liquid at the front-end, said multistage hydrolysis process further involving, between hydrolysis stages, the transfer of solid fractions towards the back-end and the transfer of liquid fractions towards the front-end, wherein after each hydrolysis stage, said biomass is shifted as solid fractions SF in a stepwise fashion towards the back-end, said aqueous solution is shifted as liquid fractions LF in a stepwise fashion towards the front-end, such that liquid fractions LF closer to the front-end are more concentrated in sugar(s) than liquid fractions closer to the back-end, and such that liquid fractions LF closer to the front-end, come into contact with solid fractions that have been less digested by enzyme than solid fractions closer to the back-end, and such that solid fractions closer to the back-end have been more digested by enzyme than solid fractions closer to the front-end, and solid fractions closer to the backend come into contact with liquid fractions that contain less sugar(s) than liquid fractions at the front-end of the process, and wherein said multistage hydrolysis process involves a repeated performance of hydrolysis stages, preferably as many hydrolysis stages as there are hydrolysis vessels in said multistage hydrolysis process.

In one embodiment the inventions relates to a method of producing glucose from a cellulosic biomass using enzymatic hydrolysis, said method comprising the steps:
a) providing n hydrolysis vessels HV₁, HV₂, HV₃, . . . , HVₙ₋₁, HVₙ, n being an integer from 2 to 100, preferably 2-50, more preferably 2-20, even more preferably 2-10,
b) adding to each hydrolysis vessel, in any order, a defined amount of cellulosic biomass, a defined volume of water and a defined amount of enzyme,
c) performing an enzymatic hydrolysis stage in all n hydrolysis vessels HV₁, HV₂, HV₃, . . . , HVₙ,
d) separating, for each hydrolysis vessel, a solid fraction from a liquid fraction, such separation resulting in solid fraction SF₁, SF₂, S₃, . . . , SFₙ₋₁, and SFₙ and in liquid fractions LF₁, LF₂, LF₃, . . . , LFₙ₋₁, and LFₙ, said solid fractions after separating preferably being in the hydrolysis vessels, and said liquid fractions after separating preferably being separate from said hydrolysis vessels,
e) discarding all or a specified partial amount of said solid fraction SFₙ,
f) transferring all or a specified amount of said fraction SFₙ₋₁, which is preferably located in said hydrolysis vessel HVₙ₋₁, into hydrolysis vessel HVₙ,
g) performing step f) for all remaining solid fractions SFₙ₋₂-SF₁, if any, which are preferably located in hydrolysis vessel HVₙ₋₂-HV₁, respectively, by transferring said remaining solid fractions into hydrolysis vessels HVₙ₋₁-HV₂, respectively
h) adding a specified amount of cellulosic biomass to hydrolysis vessel HV₁
i) taking all or a specified partial volume of liquid fraction LF₁, preferably from hydrolysis vessel HV₁, if not already separate therefrom, and storing it as final product, said final product containing sugar(s) dissolved in said liquid fraction LF₁
j) adding all or a specified partial volume of liquid fraction LF₂ to hydrolysis vessel HV₁
k) performing step j) for all remaining liquid fractions LF₃-LFₙ, if any, by adding them to hydrolysis vessels HV₂-HVₙ₋₁, respectively
l) adding a specified volume of water to hydrolysis vessel HVₙ
m) repeating steps c)-l) n−1 times wherein, for each repetition, additional enzyme is added to hydrolysis vessel HV₁, wherein, preferably, for each repetition, additional enzyme is added to hydrolysis vessel HV₁ only.

In one embodiment, said specified partial amount discarded in step e) and said specified partial amounts transferred in steps f)-g) and said specified amount added in step h) are the same.

In one embodiment, said specified partial volumes of steps i)-k) and said specified volume of step l) are the same.

In one embodiment, said specified partial amounts of steps e)-g) and said specified amount of step h), respectively, is 0.5-100% of the defined amount of cellulosic biomass added to each hydrolysis vessel during step b).

In one embodiment, said specified partial volumes of steps i)-k) and said specified volume of step l), respectively, is 0.5%-100% of the defined volume added to each hydrolysis vessel during step b).

In one embodiment, each hydrolysis stage is performed for a time period of 0.5-36 hours and/or at a temperature of 40° C.-60° C., preferably 45° C.-57° C., more preferably 48° C.-55° C.

In one embodiment, said defined amount of enzyme added initially in step b) to each hydrolysis vessel is in the range of from 0.01 wt. %-5 wt. %, preferably 0.1 wt. %-3 wt. %, more preferably 0.5-1.5% with reference to the weight of dry biomass in said vessel.

In one embodiment, said additional enzyme added to hydrolysis vessel HVi in step m) for each repetition is in the range of from 0.5wt. % to 1.5 wt. %, with reference to the weight of dry biomass in said hydrolysis vessel HV₁.

In one embodiment, the solid contents during the hydrolysis in each hydrolysis vessel is in the range of from 12% to 33%, preferably 15% - 25%, more preferably, 18% to 23%.

In one embodiment, during any of the discarding and transfer steps e) to g), the specified partial amount transferred or discarded is in the range of from 5% to 100% of said respective solid fraction, e. g. 5%, 10%, 20%, 30%, 40%, 50%, 60%, 70%, 80%, 90%, 95% and 100% and any value in between of said respective solid fraction.

In one embodiment for each hydrolysis stage, the pH is adjusted in the respective hydrolysis vessel to the working range of the respective enzyme employed, preferably to a pH in the range of from 4-7.5, preferably 4.5-7, more preferably 4.8-5.5.

In one embodiment, n is an integer with n=3-20, preferably 3-15, more preferably 3-10, even more preferably 3-4.

In one embodiment, the enzyme used for hydrolysis is selected from cellulase, hemicellulase, cellulase mixtures, hemicellulase mixtures and combinations of any of the foregoing.

In one embodiment, in step d) said separating is performed by centrifugation, filtration, sieving, decantation, pressing, plate and frame filtration, vacuum filter belt, basket centrifugation, disk stack centrifugation, and sedimentation. On an industrial scale, centrifugation or plate and frame filtration are the preferred liquid solid separation technologies to be used.

In a further aspect, the invention relates to a device for performing the method according to the present invention, said device comprising a plurality of hydrolysis vessels, preferably serially arranged, means to add cellulosic biomass, water and enzyme to each hydrolysis vessel, means to perform an enzymatic hydrolysis stage in each hydrolysis vessel, means to add and remove solid fractions and liquid fractions from each hydrolysis vessel, means to separate a solid fraction from a liquid fraction for each hydrolysis vessel, means to transfer solid fractions or parts thereof from one hydrolysis vessel to another, means to transfer liquid fractions or parts thereof from one hydrolysis vessel to another, means to add enzyme to one or several hydrolysis vessels, and means to store liquid fractions or parts thereof as final product. In one embodiment, this device also comprises a computer allowing a user to perform the method according to the present invention, which computer controls the performance of the steps of the method according to the present invention. In one embodiment, said computer allows a user to define parameters of the method according to the present invention, such as, but not limited to number and duration of hydrolysis steps, temperature of the hydrolysis step(s), amount of cellulosic biomass, water, enzyme, solid fraction(s), liquid fraction(s) that is added or removed or transferred, respectively, during each step.

The inventors have surprisingly found that by performing a multistage counter current enzymatic hydrolysis process, a substantial increase in sugar concentration can be achieved in the final product whilst at the same time, the enzyme dose used for such hydrolysis can be reduced. Also, the biomass hydrolysis time can be reduced. Overall, the process is more efficient, consumes less enzyme and results in higher product concentrations.

According to one embodiment of the method according to the present invention, there is provided a multistage hydrolysis process which is performed in a plurality of hydrolysis vessels. In such plurality of hydrolysis vessel, a plurality of hydrolysis reactions are performed in parallel, and after conclusion of such parallel performed hydrolysis reactions, a transfer of components takes place in such a manner that solid fractions and liquid fractions are moved from one hydrolysis vessel to another in opposite directions. If one considers the plurality of hydrolysis vessels as constituting a multistage process with a front-end and a back-end, such front-end being represented by the first hydrolysis vessel and the back-end being represented by the last hydrolysis vessel, in such multistage hydrolysis process, there is a repeated feeding of cellulosic biomass and enzyme at the front end, a repeated removal of enzymatically treated biomass at the back-end, a repeated addition of water at the back-end, a repeated removal of aqueous liquid at the front-end, and a repeated transfer of solid fractions towards the back-end and a repeated transfer of liquid fractions towards the front-end. Such a multi stage hydrolysis process in a plurality of hydrolysis vessels, involving the repeated transfer of solid fractions and liquid fractions in opposite directions is sometimes herein also referred to as “continuous counter current enzymatic hydrolysis”. What is removed at the back-end are the remains of the solid fractions which have been digested by the enzyme(s). What is removed at the front-end is an aqueous solution containing dissolved sugar(s), mainly in the form of glucose and xylose, resulting from the digestion of the cellulosic biomass by the appropriate enzymes. This aqueous sugar solution is then subsequently used for further downstream processing purposes. In an embodiment of the present invention, fresh, i. e. enzymatically non-digested cellulosic biomass is fed into the process in the first hydrolysis vessel, i. e. a the front-end of the process, and digested cellulosic biomass, i. e. cellulosic biomass which has been exposed to enzymatic hydrolysis is removed at the back-end, i. e. typically from the last hydrolysis vessel. The number of hydrolysis vessels may vary and typically is in the range of from 2-100, preferably 2-50, more preferably 2-20, even more preferably 2-10, even more preferably 3-6, and most preferably 4.

In one embodiment, the cellulosic biomass which is undergoing enzymatic hydrolysis in the method according to the present invention is a cellulosic biomass that has previously undergone a treatment by phosphoric acid or other mineral acid so as to separate the lignin-components from the cellulosic and hemicellulosic components. Processes for such pretreatment of cellulosic biomass are disclosed e.g. in WO 2007/111605 and WO 2009/114843. In both documents, the lignocellulosic biomass is exposed to an acidic solvent to perform a solvation and/or dissolution process subsequent to which the resultant product is transferred into a separate reactor or several reactors to be further processed. The process results in a pretreated cellulosic biomass which can then be subjected to the method according to the present invention. Such procedure for pretreatment is sometimes also referred to as cellulose solvent- and solvent-based lignocellulose fractionation (COSLIF).

In embodiments according to the present invention, for enzymatic hydrolysis, an enzyme is used selected from cellulase, hemicellulase, mixtures of different cellulases and/or hemicellulases and combinations thereof. There are numerous sources for such enzymes and may be obtained from diverse microorganisms, such as bacteria, and fungi. The cellulases and other suitable enzymes may also be recombinant enzymes which have been optimized for their function. For each hydrolysis stage, in embodiments according to the present invention, the pH is adjusted to the preferred working range of the enzyme(s) used. Typically, such working range is in the range of from 4-7.5, preferably 4.5-7, more preferably 4.8-5.5. pH adjustment can be achieved by any suitable means, such as addition of a base or acid, as necessary, but also through the use of an appropriate buffer system. Suitable bases for adjusting the pH are selected from ammonium hydroxide, sodium hydroxide, potassium hydroxide, urea, lime, calcium hydroxide, sodium carbonate, potassium carbonate and others. Suitable buffer systems are known to persons skilled in the art, and useful examples are ammonium hydroxide, potassium hydroxide, sodium hydroxide, citrate buffer, and phosphate buffers.

In embodiments according to the present invention, first there is an enzymatic hydrolysis performed in all n hydrolysis vessels, and subsequently, there is a transfer of components. Preferably, the transfer of components is initiated by separating, for each hydrolysis vessel, a solid fraction from a liquid fraction. Such separation can be achieved by various techniques, including, but not limited to centrifugation, filtration, decantation, pressing, sieving, plate and frame filtration, vacuum filter belt, basket centrifugation, disk stack centrifugation, and sedimentation. On the industrial scale, centrifugation or plate and frame filtration are the most likely liquid solid separation technologies to be used. Such separation may be performed in such a manner that the solid fraction remains in the respective hydrolysis vessel whereas the liquid fraction is retained separately from each respective hydrolysis vessel. This would be typically the case if the separation occurs by centrifugation. Alternatively, the separation may, however, also be performed such that the liquid fraction remains in the hydrolysis vessel, whereas the solid fraction is obtained separate therefrom. In further embodiments, the whole hydrolysis reaction, after completion at the end of the hydrolysis stage, is transferred into a separate vessel, and the two fractions, i. e. the solid fraction and the liquid fraction are obtained separate from and outside of the respective hydrolysis vessel.

Thereafter, in embodiments, at the back-end of the hydrolysis process, i. e. at the last hydrolysis vessel, the entire solid fraction or a part thereof is removed therefrom and discarded, and the other solid fractions resulting from hydrolysis in the other hydrolysis vessels are used to serially replenish the back-end of the hydrolysis vessel system. Effectively, all solid fractions move up one position by one hydrolysis vessel, and the first hydrolysis vessel is filled/replenished with fresh, i. e. enzymatically undigested cellulosic biomass. It should be noted, however, that during the transfer just described, there may also occur only a partial transfer, in that only a part of each solid fraction is transferred to the next hydrolysis vessel. Such part may be a suitable percentage of the entire respective solid fraction, e. g. 5%, 10%, 15%, 20%, 25%, 30%, 35%, 40%, 45%, 50%, 55%, 60%, 65%, 70%, 75%, 80%, 85%, 90%, 95%, and any intervening percentage. In embodiments of the present invention, the solid fractions or defined parts thereof from each hydrolysis vessel are transferred upstream by one hydrolysis vessel at a time, and the respective liquid fractions or defined parts thereof are transferred downstream one hydrolysis vessel at a time. Typically, the amounts transferred upstream are the same with respect to each other. In a further embodiment, the volumes transferred downstream are, in one embodiment, the same with respect to each other.

In one embodiment at the front-end, i. e. the first hydrolysis vessel, the liquid fraction is used as the final product and represents an aqueous solution of sugar(s) typically glucose, but also, possibly, pentose(s). The liquid fraction from the second hydrolysis vessel is used to replenish the first hydrolysis vessel, the liquid fraction or parts thereof form the third hydrolysis vessel is used to replenish the second hydrolysis vessel, and the liquid fraction of the nth hydrolysis vessel is used to replenish the n-lth hydrolysis vessel. Into the nth hydrolysis vessel, water is added, preferably, at the same volume that was used to replenish the n-lth hydrolysis vessel. With the solid fractions having moved upstream by one hydrolysis vessel and the liquid fractions having moved downstream by one hydrolysis vessel, the system is ready for the next hydrolysis stage which is, again, performed for a defined period of time, typically in the range of from 0.5 h-36 h and/or at a temperature of 40° C.-60° C., preferably 45° C.-57° C., more preferably 48° C.-55° C., even more preferably at the optimum temperature of the respective enzyme that is used for the enzymatic hydrolysis.

In one embodiment, the amount of enzyme that is initially used for the first enzymatic hydrolysis is in the range of from 0.01 wt. %-5 wt. %, preferably 0.1 wt. %-3 wt. %, more preferably 0.5 wt. %-1.5 wt. % , with reference to the weight of dry biomass in said vessel.

It should be noted that in embodiments according to the present invention, for each subsequent hydrolysis stage, fresh enzyme is added only in the first hydrolysis vessel. This is, because the present inventors believe, without wishing to be bound by any theory, that the enzyme is at least partially bound or adsorbed by the solid fraction and is therefore at least partially transferred along during each upstream transfer of solid fractions. The amount of enzyme added in the second, third and any subsequent hydrolysis stage is in the range of from 0.5wt. % to 1.5 wt. %, with respect to the weight of dry biomass in the first hydrolysis vessel.

In embodiments according to the present invention, the solids content in each hydrolysis vessel is in the range of from 12% to 33%. Thus, using the method according to the present invention, a high solids content can be used which thereby contributes to the good yield in final product(s).

In embodiments according to the present invention, the number of hydrolysis stages varies from n=3-n=20, preferably n=3-n=15, more preferably n=3-n=10, even more preferably n=3-n=6, and most preferably n=3-n=4.

In one embodiment of the method according to the present invention, the number of hydrolysis stages and vessels n is 3 or 4, the solids content in each hydrolysis vessel is 18% to 21%, the amount of enzyme added initially and in subsequent hydrolysis stages is in the range of from 1.0 wt. % to 1.5 wt. %, with respect to the weight of dry biomass in the first hydrolysis vessel.

As used herein, the term “dry solids transfer”, also abbreviated as “DST” refers to the percentage of solids transferred from one hydrolysis vessel to the next between two hydrolysis stages As used herein, the term “biomass residence time”, also abbreviated as “BRT” refers to the amount of time the biomass spent in each reactor from its input at the front-end to its exit at the back-end.

Furthermore, reference is made now to the figures wherein

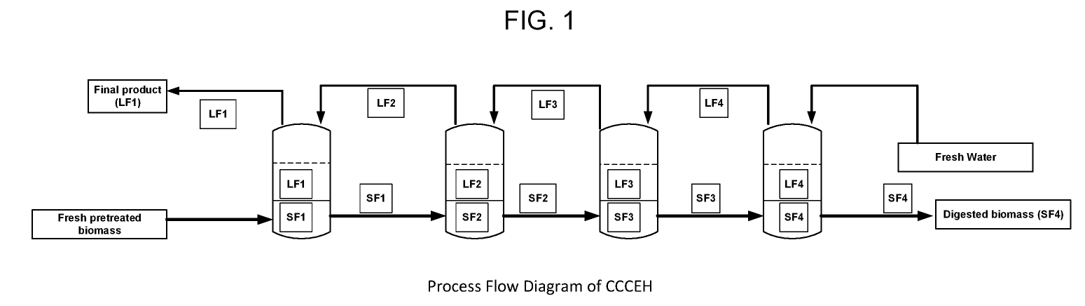

FIG. 1 shows a flow diagram for a multi-stage continuous counter current enzymatic hydrolysis (herein also sometimes abbreviated as CCCEH) in accordance with an embodiment of the present invention, showing few hydrolysis vessels.

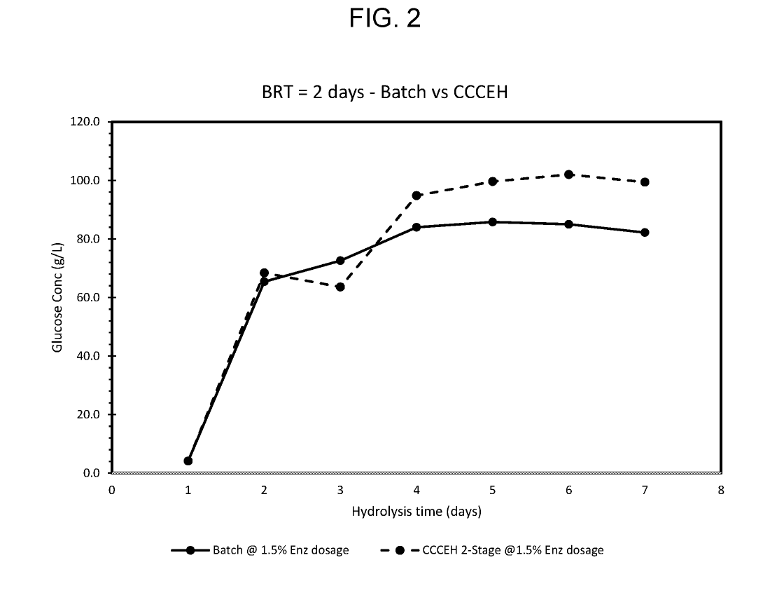

FIG. 2 shows a batch hydrolysis versus a 2-stage counter current enzymatic hydrolysis with a biomass residence time of 2 days (48 h) at 18% solids content and 1.5% wt enzyme dosage

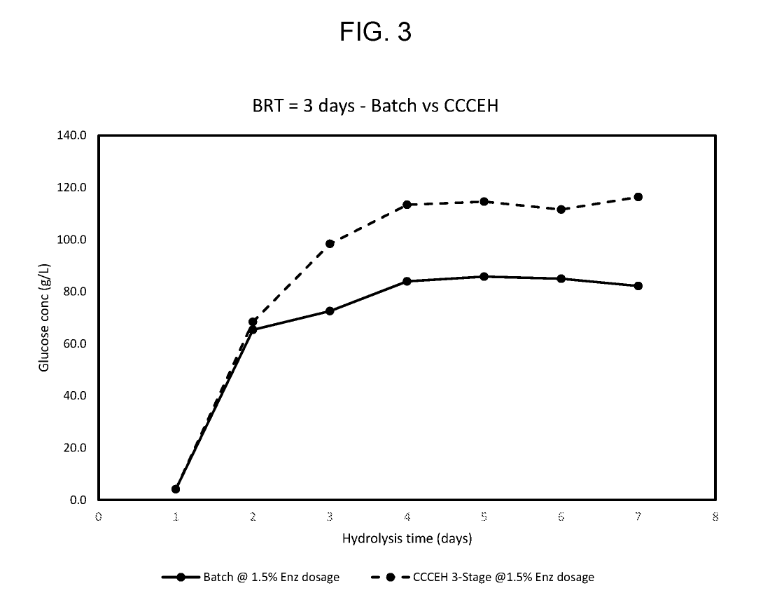

FIG. 3 shows a batch hydrolysis versus a 3-stage counter current enzymatic hydrolysis with a biomass residence time of 3 days (72 h) at 18% solids content and 1.5% wt enzyme dosage

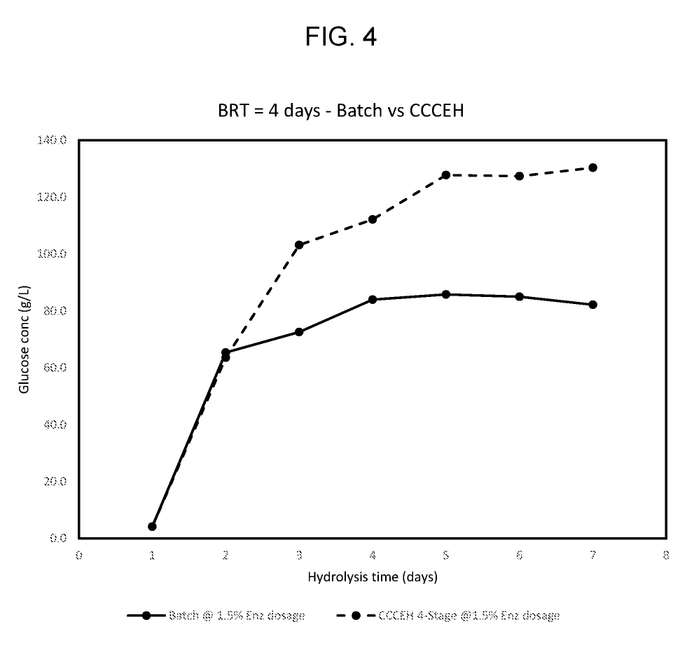

FIG. 4 shows a batch hydrolysis versus a 4-stage counter current enzymatic hydrolysis with a biomass residence time of 4 days (96 h) at 18% solids content and 1.5% wt enzyme dosage

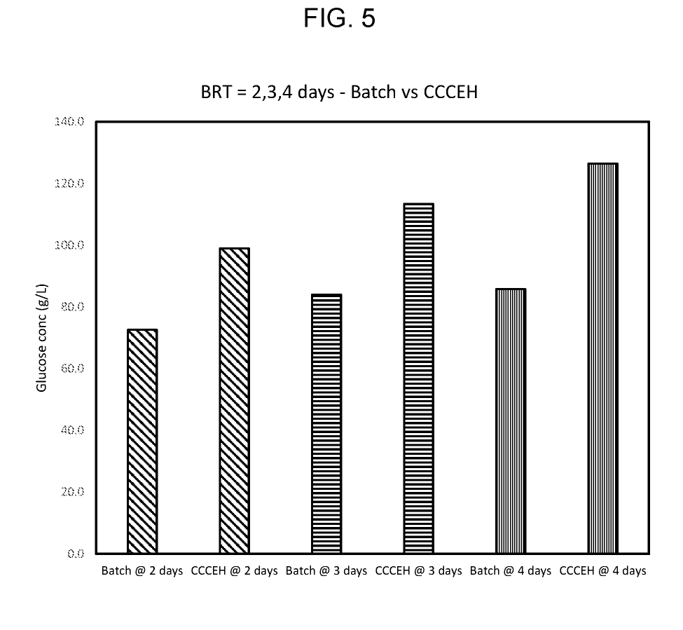

FIG. 5 shows a summary of batch hydrolysis versus 2,3,4-stage counter current enzymatic hydrolysis at 18% solids content and 1.5% wt enzyme dosage

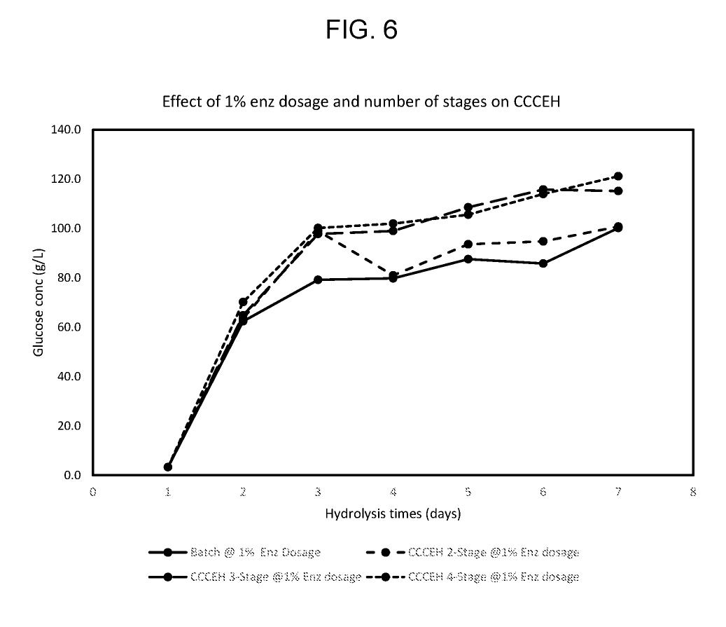

FIG. 6 shows a summary of batch hydrolysis versus 2,3,4-stage counter current enzymatic hydrolysis at 18% solids content and 1.0% wt enzyme dosage

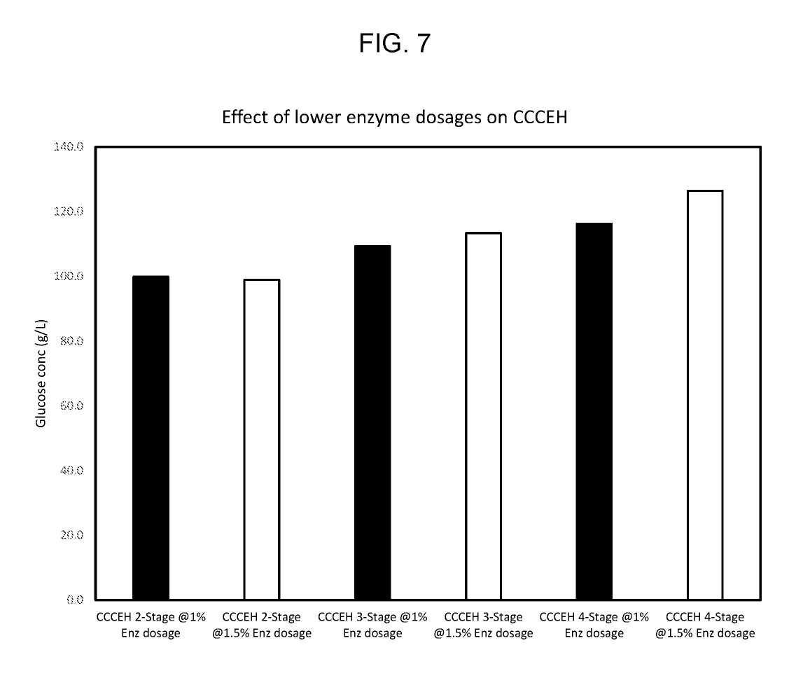

FIG. 7 shows a summary of 2,3,4-stage counter current enzymatic hydrolysis at 18% solids content with 1.0% wt and 1.5% wt enzyme dosage

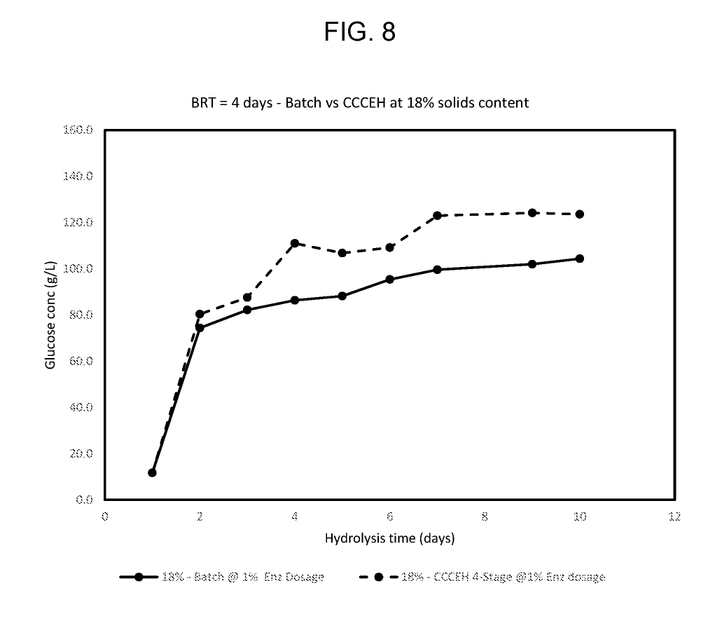

FIG. 8 shows a summary of a batch hydrolysis versus a 4-stage counter current enzymatic hydrolysis at 18% solids content at 1.0% wt enzyme dosage

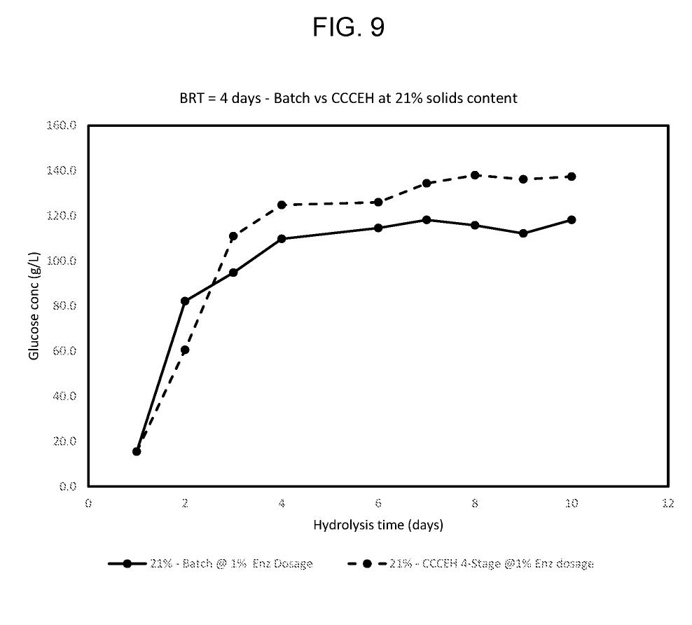

FIG. 9 shows a summary of a batch hydrolysis versus a 4-stage counter current enzymatic hydrolysis at 21% solids content at 1.0% wt enzyme dosage.

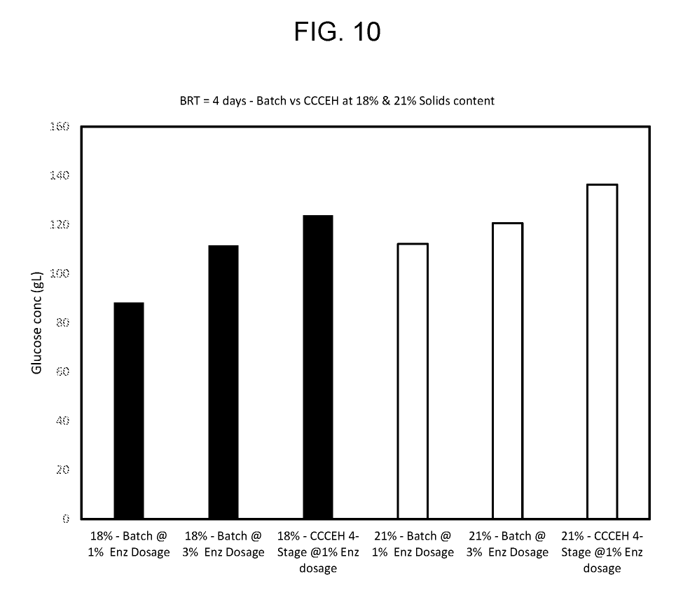

FIG. 10 shows a summary of batch hydrolysis versus 4-stage counter current enzymatic hydrolysis at 18% solids content, 21% solids content, 1.0% wt enzyme dosage and 3.0% wt enzyme dosage.

Furthermore, reference is made to the examples which are given to illustrate but not to limit the present invention:

### EXAMPLES

### Example 1

Effect of a biomass residence time (BRT) of 2 days on cellulose digestibility: 100% Dry Solids Transfer (100% DST) and 24 h biomass transfer in a 2-stage counter current enzymatic hydrolysis

Procedure

Pretreatment of Cellulosic Biomass

Mixed hardwood say dust was pretreated using the COSLIF procedure and process licensed from Virginia Tech (Percival Zhang). 30 kg of hardwood saw dust were presoaked with 150 kg of 85% of H₃O₄ at room temperature for 60 minutes. The presoaked biomass was them continuously mixed in a 200 gallon planetary mixer/reactor (Charles Ross and Sons. Hauppauge, N.Y.) equipped with anchor style impellors and a disperser blade) and heated at 70° C. using a recirculating hot water system (Thermal Care Aqua RQ Series heater Niles, Ill.) for 60 minutes. The pretreatment was quenched by adding 300 kg of 95% ethanol in the reactor, the resulting slurry was pumped into a plate and frame filtration system where the pretreated biomass was washed via counter current washing with 524 kg of 95% ethanol followed by 866 kg of water. After the washing process the washed pretreated biomass was pressed in a hydraulic press to elevate the solids to approximately 38%. The washed pretreated biomass (with a moisture content of approximately 58%) was then conveyed into hydrolysis vessels where various counter current enzymatic hydrolysis experiments were performed.

Hydrolysis procedure

173 g of pretreated biomass containing approximately 42% solids (72 g dry pretreated biomass) and 226 g of water were added to three hydrolysis vessels labeled HV1, HV2, and HV Control; the pH was adjusted with ammonium hydroxide to 5.3; 1.1 g of enzyme was then added and the mixtures were incubated under mixing at 200 rpm at 50° C. for 24 hours. The final solids content is all hydrolysis vessels was 18% and the enzyme dose used was 1.5% wt (15 mg enzyme per g of dry biomass). After 24 hours the hydrolysis vessels HV1 and HV2 were centrifuged, the liquid fractions labeled LF1 (133 g) and LF2 (223 g) were separated from the solids fractions SF1 (72 g dry), and SF2 (72 g dry) that remained in their respective hydrolysis vessels.

A solids transfer was performed as follows:
1. 173 g of fresh moist pretreated biomass containing approximately 42% solids (72 g dry pretreated biomass) were added to HV1;
2. 72 g dry digested biomass (SF1) were removed from HV1 and transferred to HV2;
3. 72 g dry digested biomass (SF2) were removed from HV2 and frozen for subsequent analyses.

A liquid transfer was performed as follows:

1. 226 g of fresh water were added to HV2;

2. 223 g of LF2 were transferred to HV1;

3. 133 g of LF1 were analyzed for their glucose content and frozen for subsequent analyses.

The pH in all three vessels was adjusted back to 5.3 with ammonium hydroxide; 1.1 g of make-up enzyme was then added to only HV1 (bringing the enzyme dose back to 1.5% wt) and the mixtures were incubated under mixing at 200 rpm at 50° C. for 24 hours. The transfer procedure described above was repeated every 24 hours until the glucose content in liquid fraction LF1 from HV1 remained constant. The procedure was performed for 14 days, and a steady state was reached after 4-5 days.

Results

FIG. 2 shows that the glucose concentration achieved with the batch enzymatic hydrolysis after 2 days was 73 g/L whereas steady state was achieved for the counter current enzymatic hydrolysis with 2 days biomass residence time (BRT) after approximately 5 days with an average glucose concentration of 99 g/L.2-stage continuous counter current enzymatic hydrolysis shows a 36% increase in glucose concentration compared to batch hydrolysis.

### Example 2

Effect of biomass residence time (BRT) of 3 days on cellulose digestibility: 100% Dry Solids Transfer (100% DST) and 24 h biomass transfer in a 3-stage counter current enzymatic hydrolysis

Procedure

173 g of pretreated biomass containing approximately 42% solids (72 g dry pretreated biomass) and 226 g of water were added to four hydrolysis vessels labeled HV1, HV2, HV3 and HV Control; the pH was adjusted with ammonium hydroxide to 5.3; 1.1 g of enzyme was then added and the mixtures were incubated under mixing at 200 rpm at 50° C. for 24 hours. The final solids content is all hydrolysis vessels was 18% and the enzyme dose used was 1.5% wt (15 mg enzyme per g of dry biomass. After 24 hours the hydrolysis vessels HV1, HV2 and HV3 were centrifuged, the liquid fractions labeled LF1 (203 g), LF2 (245 g) and LF3 (233 g) were separated from the solids fractions SF1 (72 g dry), SF2 (72 g dry) and SF3 (72 g dry) that remained in their respective hydrolysis vessels.

The solids transfer was performed as follows:
1. 173 g of fresh moist pretreated biomass containing approximately 42% solids (72 g dry pretreated biomass) were added to HV1;
2. 72 g dry digested biomass (SF1) were removed from HV1 and transferred to HV2;
3. 72 g dry digested biomass (SF2) were removed from HV2 and transferred to HV3;
4. 72 g dry digested biomass (SF3) were removed from HV3 and frozen for subsequent analyses.

The liquid transfer was performed as follows:

1. 226 g of fresh water were added to HV3;

2. 233 g of LF3 were transferred to HV2;

3. 245 g of LF2 were transferred to HV1;

4. 203 g of LF1 were analyzed for their glucose content and frozen for subsequent analyses.

The pH in all four vessels was adjusted back to 5.3 with ammonium hydroxide; 1.1 g of make-up enzyme was then added to only HV1 (bringing the enzyme dose back to 1.5% wt) and the mixtures were incubated under mixing at 200 rpm at 50° C. for 24 hours. The transfer procedure described above was repeated every 24 hours until the glucose content in liquid fraction LF1 from HV1 remained constant.

Results

FIG. 3 shows that the glucose concentration achieved with the batch enzymatic hydrolysis after 3 days was 84 g/L whereas steady state was achieved for the counter current enzymatic hydrolysis with 3 days biomass residence after approximately 4 days with an average glucose concentration of 113 g/L. 3-stage continuous counter current enzymatic hydrolysis show a 35% increase in glucose concentration compare to batch hydrolysis

### Example 3

Effect of biomass residence time (BRT) of 4 days on cellulose digestibility: 100% Dry Solids Transfer (100% DST) and 24h biomass transfer in a 4-stage counter current enzymatic hydrolysis

Procedure

173 g of pretreated biomass containing approximately 42% solids (72 g dry pretreated biomass) and 226 g of water were added to five hydrolysis vessels labeled HV1, HV2, HV3 and HV Control; the pH was adjusted with ammonium hydroxide to 5.3; 1.1 g of enzyme was then added and the mixtures were incubated under mixing at 200 rpm at 50° C. for 24 hours. The final solids content is all hydrolysis vessels was 18% and the enzyme dose used was 1.5% wt (15 mg enzyme per g of dry biomass). After 24 hours the hydrolysis vessels HV1, HV2 and HV3 were centrifuged, the liquid fractions labeled LF1 (200 g), LF2 (257 g) LF3 (239 g) and LF4 (237 g) were separated from the solids fractions SF1 (72 g dry), SF2 (72 g dry), SF3 (72 g dry) and SF4 (72 g dry) that remained in their respective hydrolysis vessels.

The solids transfer was performed as follows:
1. 173 g of fresh moist pretreated biomass containing approximately 42% solids (72 g dry pretreated biomass) were added to HV1;
2. 72 g dry digested biomass (S1F) were removed from HV1 and transferred to HV2;
3. 72 g dry digested biomass (SF2) were removed from HV2 and transferred to HV3;
4. 72 g of dry digested biomass (SF3) were removed from HV3 and transferred to HV4
5. 72 g dry digested biomass (SF4) were removed from HV4 and frozen for subsequent analyses.

The liquid transfer was performed as follows:

1. 226 g of fresh water were added to HV4;

2. 237 g of LF4 were transferred to HV3;

3. 239 g of LF3 were transferred to HV2;

4. 257 g of LF2 was transferred to HV1

5. 200 g of LF1 were analyzed for their glucose content and frozen for subsequent analyses.

The pH in all four vessels was adjusted back to 5.3 with ammonium hydroxide; 1.1 g of make-up enzyme was then added to only HV1 (bringing the enzyme dose back to 1.5% wt) and the mixtures were incubated under mixing at 200 rpm at 50° C. for 24 hours. The transfer procedure described above was repeated every 24 hours until the glucose content in liquid fraction LF1 from HV1 remained constant.

Results

FIG. 4 shows that the glucose concentration achieved with the batch enzymatic hydrolysis after 4 days was 86 g/L whereas steady state was achieved for the counter current enzymatic hydrolysis with 4 days biomass residence after approximately 5 days with an average glucose concentration of 127 g/L. 4-stage continuous counter current enzymatic hydrolysis show a 47% increase in glucose concentration compare to batch hydrolysis.

### Example 4

Effect of number of stages on counter current enzymatic hydrolysis

Procedure

Same as examples 1-3

Results

The following findings are summarized in FIG. 5
1. A 15% increase in glucose concentration was achieved with continuous counter current enzymatic hydrolysis when the number of stages were increased from 2 to 3.
2. A 12% increase in glucose concentration was achieved with continuous counter current enzymatic hydrolysis when the number of stages were increased from 3 to 4.
3. A 28% increase in glucose concentration was achieved with continuous counter current enzymatic hydrolysis when the number of stages were increased from 2 to 4.

### Example 5

Effect of lower enzyme dosages on 2,3,4-stages counter current enzymatic hydrolysis

Procedure

The initial enzyme added in all hydrolysis vessel at the beginning of enzymatic hydrolysis was 0.72 g. The make-up enzyme added in HV1 after each transfer was 0.72 g. The rest of the procedure was similar to examples 1-3.

Results

FIG. 6 shows that the glucose concentration achieved with the batch enzymatic hydrolysis after 2, 3 and 4 days was respectively 79, 80, and 88 g/L whereas steady state was achieved for the counter current enzymatic hydrolysis with respectively 2, 3 and 4 days biomass residence after 7, 6 and 6 days with an average glucose concentration of respectively 100, 109 and 116 g/L.

2-stage continuous counter current enzymatic hydrolysis show a 26% increase in glucose concentration compared to batch hydrolysis.

3-stage continuous counter current enzymatic hydrolysis show a 37% increase in glucose concentration compared to batch hydrolysis.

4-stage continuous counter current enzymatic hydrolysis show a 33% increase in glucose concentration compared to batch hydrolysis.

The following findings are summarized in FIG. 7:
1. Little to no impact on the percent increase in glucose was observed with a 33% reduction in enzyme dosage for the lower number of stages in continuous counter current enzymatic hydrolysis.
2. No percent increase in glucose was observed when the enzyme dosage was increased from 1% wt to 1.5% wt in 2-stage counter current enzymatic hydrolysis.
3. 4% increase in glucose was observed when the enzyme dosage was increased from 1% wt to 1.5% wt in 3-stage counter current enzymatic hydrolysis.
4. 9%increase in glucose was observed when the enzyme dosage was increased from 1% wt to 1.5% wt in 4-stage counter current enzymatic hydrolysis.

### Example 6

Effect of solids content in a 4-Stage counter current enzymatic hydrolysis at 1.0% wt enzyme dosage.

Procedure

Procedure for 18% solids content counter current enzymatic hydrolysis 147 g of pretreated biomass containing approximately 49% solids (72 g dry pretreated biomass) and 252 g of water were added to five hydrolysis vessels labeled HV1, HV2, HV3 and HV Control; the pH was adjusted with ammonium hydroxide to 5.3; 0.72 g of enzyme was then added and the mixtures were incubated under mixing at 200 rpm at 50° C. for 24 hours. The final solids content is all hydrolysis vessels was 18% and the enzyme dose used was 1.5% wt (15 mg enzyme per g of dry biomass). After 24 hours the hydrolysis vessels HV1, HV2 and HV3 were centrifuged, the liquid fractions labeled LF1 (211 g), LF2 (210 g) LF3 (207 g) and LF4 (209 g) were separated from the solids fractions SF1 (72 g dry), SF2 (72 g dry), SF3 (72 g dry) and SF4 (72 g dry) that remained in their respective hydrolysis vessels.

The solids transfer was performed as follows:
1. 147 g of fresh moist pretreated biomass containing approximately 49% solids (72 g dry pretreated biomass) were added to HV1;
2. 72 g dry digested biomass (S1F) were removed from HV1 and transferred to HV2;
3. 72 g dry digested biomass (SF2) were removed from HV2 and transferred to HV3;
4. 72 g of dry digested biomass (SF3) were removed from HV3 and transferred to HV4
5. 72 g dry digested biomass (SF4) were removed from HV4 and frozen for subsequent analyses.

The liquid transfer was performed as follows:
1. 252 g of fresh water were added to HV4;
2. 209 g of LF4 were transferred to HV3;
3. 207 g of LF3 were transferred to HV2;
4. 209 g of LF2 was transferred to HV1
5. 211 g of LF1 were analyzed for their glucose content and frozen for subsequent analyses.

The pH in all four vessels was adjusted back to 5.3 with ammonium hydroxide; 0.72 g of make-up enzyme was then added to only HV1 (bringing the enzyme dose back to 1.5% wt) and the mixtures were incubated under mixing at 200 rpm at 50° C. for 24 hours. The transfer procedure described above was repeated every 24 hours until the glucose content in liquid fraction LF1 from HV1 remained constant.

Procedure for 21% solids content counter current enzymatic hydrolysis 171 g of pretreated biomass containing approximately 49% solids (84g dry pretreated biomass) and 228 g of water were added to five hydrolysis vessels labeled HV1, HV2, HV3 and HV Control; the pH was adjusted with ammonium hydroxide to 5.3; 0.84 g of enzyme was then added and the mixtures were incubated under mixing at 200 rpm at 50° C. for 24 hours. The final solids content is all hydrolysis vessels was 21% and the enzyme dose used was 1.5% wt (15 mg enzyme per g of dry biomass). After 24 hours the hydrolysis vessels HV1, HV2 and HV3 were centrifuged, the liquid fractions labeled LF1 (186 g), LF2 (185 g) LF3 (189 g) and LF4 (192 g) were separated from the solids fractions SF1 (84 g dry), SF2 (84 g dry), SF3 (84 g dry) and SF4 (84 g dry) that remained in their respective hydrolysis vessels.

The solids transfer was performed as follows:
1. 171 g of fresh moist pretreated biomass containing approximately 49% solids (84 g dry pretreated biomass) were added to HV1;
2. 84 g dry digested biomass (S1F) were removed from HV1 and transferred to HV2;
3. 84 g dry digested biomass (SF2) were removed from HV2 and transferred to HV3;
4. 84 g of dry digested biomass (SF3) were removed from HV3 and transferred to HV4
5. 84 g dry digested biomass (SF4) were removed from HV4 and frozen for subsequent analyses.

The liquid transfer was performed as follows:
1. 228 g of fresh water were added to HV4;
2. 192 g of LF4 were transferred to HV3;
3. 189 g of LF3 were transferred to HV2;
4. 185 g of LF2 was transferred to HV1
5. 186 g of LF1 were analyzed for their glucose content and frozen for subsequent analyses.

The pH in all four vessels was adjusted back to 5.3 with ammonium hydroxide; 0.84 g of make-up enzyme was then added to only HV1 (bringing the enzyme dose back to 1.5% wt) and the mixtures were incubated under mixing at 200 rpm at 50° C. for 24 hours. The transfer procedure described above was repeated every 24 hours until the glucose content in liquid fraction LF1 from HV1 remained constant.

Results

FIG. 8 shows that the glucose concentration achieved with the batch enzymatic hydrolysis at 18% solids content after 4 days was 88 g/L whereas steady state was achieved for the counter current enzymatic hydrolysis at 18% solids content with 4 days biomass residence after approximately 7 days with an average glucose concentration of 124 g/L. 4-stage continuous counter current enzymatic hydrolysis shows a 40% increase in glucose concentration compared to batch hydrolysis.

FIG. 9 shows that the glucose concentration achieved with the batch enzymatic hydrolysis at 21% solids content after 4 days was 112 g/L whereas steady state was achieved for the counter current enzymatic hydrolysis at 21% solids content with 4 days biomass residence after approximately 7 days with an average glucose concentration of 136 g/L. 4-stage continuous counter current enzymatic hydrolysis shows a 22% increase in glucose concentration compared to batch hydrolysis.

The following findings are summarized in FIG. 10:
1. An 11% increase in glucose was achieved at 18% solids content with continuous counter current enzymatic hydrolysis by reducing the enzyme dosage in half in continuous counter current enzymatic hydrolysis while the enzyme dosage in the batch hydrolysis was 3% wt.
2. 13% increase in glucose was achieved at 21% solids content with continuous counter current enzymatic hydrolysis by reducing the enzyme dosage in half in continuous counter current enzymatic hydrolysis while the enzyme dosage in the batch hydrolysis was 3% wt.

The various samples used in examples 1-6 can be summarized in terms of their enzyme dosages and in terms of their percentage of total solids (% TS) in the following table:

TABLE
Enzyme dosages and % TS

| | Target Slurry | Target solids | Target dry | Target enzyme | Target enzyme | Actual |
|---|---|---|---|---|---|---|
| Sample | Wt (g) | content in HYD | Biomass Wt (g) | dosage (% wt) | Wt (g) | Biomass % MC |
| Example 1-4 | 400.0 | 18.0% | 72.00 | 1.5% | 1.08 | 58.4% |
| Example 5 | 400.0 | 18.0% | 72.00 | 1.0% | 0.72 | 58.4% |
| Example 6 | 400.0 | 18.0% | 72.00 | 1.0% | 0.72 | 51.0% |
| Example 6 | 400.0 | 18.0% | 72.00 | 1.5% | 1.08 | 51.0% |
| Example 6 | 400.0 | 18.0% | 72.00 | 3.0% | 2.16 | 51.0% |
| Example 6 | 400.0 | 21.0% | 84.00 | 1.0% | 0.84 | 51.0% |
| Example 6 | 400.0 | 21.0% | 84.00 | 1.5% | 1.26 | 51.0% |
| Example 6 | 400.0 | 21.0% | 84.00 | 3.0% | 2.52 | 51.0% |

| | | Actual Wet | Actual Dry | Actual | | Actual |
|---|---|---|---|---|---|---|
| | Actual | Biomass | Biomass Wt | Enzyme Wt | Actual H2O | Slurry Wt |
| Sample | Biomass % TS | Wet (g) | (g) | (g) | added (g) | (g) |
| Example 1-4 | 41.6% | 173.1 | 72.0 | 1.08 | 225.8 | 400.0 |
| Example 5 | 41.6% | 173.1 | 72.0 | 0.72 | 226.2 | 400.0 |
| Example 6 | 49.0% | 146.9 | 72.0 | 0.72 | 252.3 | 400.0 |
| Example 6 | 49.0% | 146.9 | 72.0 | 1.08 | 252.0 | 400.0 |
| Example 6 | 49.0% | 146.9 | 72.0 | 2.16 | 250.9 | 400.0 |
| Example 6 | 49.0% | 171.4 | 84.0 | 0.84 | 227.7 | 400.0 |
| Example 6 | 49.0% | 171.4 | 84.0 | 1.26 | 227.3 | 400.0 |
| Example 6 | 49.0% | 171.4 | 84.0 | 2.52 | 226.1 | 400.0 |

### Examples 1-6

### Enzymes Dosages and Total Solids Present in Biomass

“% MC” refers to moisture content. “% TS” refers to total solids. “Target solids content in HYD” refers to the amount of dry solids that will be present in the hydrolysis vessel(s) during enzymatic hydrolysis. “Biomass % TS” refers to the amount of dry matter present in the biomass after a COSLIF-pretreatment. %Total Solids (% TS)+% Moisture Content (% MC)=100%.

1. A method of producing glucose from a cellulosic biomass using enzymatic hydrolysis, wherein said enzymatic hydrolysis is performed in a multistage hydrolysis process in a plurality of hydrolysis vessels, said plurality of hydrolysis vessels being serially arranged such that there is a first hydrolysis vessel, a last hydrolysis vessel and, optionally, one or several hydrolysis vessels in between, wherein during said multistage process, each hydrolysis vessel contains a solid fraction SF which is biomass and a liquid fraction LF which is an aqueous solution, said multistage process having a front-end at said first hydrolysis vessel and a back-end at said last hydrolysis vessel, said multistage process involving an initial addition of enzyme to each hydrolysis vessel, said multi-stage hydrolysis process further involving in each hydrolysis stage the feeding of cellulosic biomass and enzyme at the front-end, the removal of enzymatically treated biomass at the back-end, the addition of water at the back-end, and the removal of aqueous liquid at the front-end, said multistage hydrolysis process further involving, between hydrolysis stages, the transfer of solid fractions towards the back-end and the transfer of liquid fractions towards the front-end, wherein after each hydrolysis stage, said biomass is shifted as solid fractions SF in a stepwise fashion towards the back-end, said aqueous solution is shifted as liquid fractions LF in a stepwise fashion towards the front-end, such that liquid fractions LF closer to the front-end are more concentrated in sugar(s) than liquid fractions closer to the back-end, and such that liquid fractions LF closer to the front-end, come into contact with solid fractions that have been less digested by enzyme than solid fractions closer to the back-end, and such that solid fractions closer to the back-end have been more digested by enzyme than solid fractions closer to the front-end, and solid fractions closer to the back-end come into contact with liquid fractions that contain less sugar(s) than liquid fractions at the front-end of the process, and wherein said multistage hydrolysis process involves a repeated performance of hydrolysis stages, preferably as many hydrolysis stages as there are hydrolysis vessels in said multistage hydrolysis process.

2. A method of producing glucose from a cellulosic biomass using enzymatic hydrolysis, in particular according to claim 1, comprising the steps:
a) providing n hydrolysis vessels HV₁, HV₂, HV₃, . . . , HVₙ₋₁, HVₙ, n being an integer from 2 to 100, preferably 2-50, more preferably 2-20, even more preferably 2-10,
b) adding to each hydrolysis vessel, in any order, a defined amount of cellulosic biomass, a defined volume of water and a defined amount of enzyme,
c) performing an enzymatic hydrolysis stage in all n hydrolysis vessels HV₁, HV₂, HV₃, . . . , HVₙ,
d) separating, for each hydrolysis vessel, a solid fraction from a liquid fraction, such separation resulting in solid fraction SF₁, SF₂, SF₃, SFₙ₋₁, and SFₙ and in liquid fractions LF₁, LF₂, LF₃, . . . , LFₙ₋₁, and LFₙ, said solid fractions after separating preferably being in the hydrolysis vessels, and said liquid fractions after separating preferably being separate from said hydrolysis vessels,
e) discarding all or a specified partial amount of said solid fraction SFₙ,
f) transferring all or a specified amount of said fraction SFₙ₋₁, which is preferably located in said hydrolysis vessel HVₙ₋₁, into hydrolysis vessel HVₙ,
g) performing step f) for all remaining solid fractions SFₙ₋₂-SF₁, if any, which are preferably located in hydrolysis vessel HVₙ₋₂-HV₁, respectively, by transferring said remaining solid fractions into hydrolysis vessels HVₙ₋₁-HV₂, respectively
h) adding a specified amount of cellulosic biomass to hydrolysis vessel HV₁
i) taking all or a specified partial volume of liquid fraction LF₁, preferably from hydrolysis vessel HV₁, if not already separate therefrom, and storing it as final product, said final product containing sugar(s) dissolved in said liquid fraction LF₁
j) adding all or a specified partial volume of liquid fraction LF₂ to hydrolysis vessel HV₁
k) performing step j) for all remaining liquid fractions LF₃-LFₙ, if any, by adding them to hydrolysis vessels HV₂-HVₙ₋₁, respectively
l) adding a specified volume of water to hydrolysis vessel HVₙ
m) repeating steps c)-l) n−1 times wherein, for each repetition, additional enzyme is added to hydrolysis vessel HV₁, wherein, preferably, for each repetition, additional enzyme is added to hydrolysis vessel HV₁ only.

3. The method according to claim 2, wherein said specified partial amount discarded in step e) and said specified partial amounts transferred in steps f)-g) and said specified amount added in step h) are the same.

4. The method according to claim 2, wherein said specified partial volumes of steps i)-k) and said specified volume of step l) are the same.

5. The method according to claim 2, wherein said specified partial amounts of steps e)-g) and said specified amount of step h), respectively, is 0.5-100% of the defined amount of cellulosic biomass added to each hydrolysis vessel during step b).

6. The method according to claim 2, wherein said specified partial volumes of steps i)-k) and said specified volume of step l), respectively, is 0.5%-100% of the defined volume added to each hydrolysis vessel during step b).

7. The method according to claim 1, wherein each hydrolysis stage is performed for a time period of 0.5-36 hours and/or at a temperature of 40° C.-60° C., preferably 45° C.-57° C., more preferably 48° C.-55° C.

8. The method according to claim 2, wherein said defined amount of enzyme added initially in step b) to each hydrolysis vessel is in the range of from 0.01 wt. %-5 wt. %, preferably 0.1 wt. %-3 wt. %, more preferably 0.5 wt. %-1.5wt. % with reference to the weight of dry biomass in said vessel.

9. The method according to claim 2, wherein said additional enzyme added to hydrolysis vessel HV₁ in step m) for each repetition is in the range of from 0.5wt. % to 1.5wt. %, with reference to the weight of dry biomass in said hydrolysis vessel HV₁.

10. The method according to claim 1, wherein the solid contents during the hydrolysis in each hydrolysis vessel is in the range of from 12% to 33%, preferably 15%-25%, more preferably 18% to 23%.

11. The method according to claim 2, wherein, during any of the discarding and transfer steps e) to g), the specified partial amount transferred or discarded is in the range of from 5% to 100% of said respective solid fraction, e. g. 5%, 10%, 20%, 30%, 40%, 50%, 60%, 70%, 80%, 90%, 95% and 100% and any value in between of said respective solid fraction.

12. The method according to claim 1, wherein for each hydrolysis stage, the pH is adjusted in the respective hydrolysis vessel to the working range of the respective enzyme employed, preferably to a pH in the range of from 4-7.5, preferably 4.5 to 7, more preferably 4.8-5.5.

13. The method according to claim 2, wherein n=3-20, preferably 3-15, more preferably 3-10, even more preferably 3-4.

14. The method according to claim 1, wherein the enzyme used for hydrolysis is selected from cellulase, hemicellulase, cellulase mixtures, hemicellulase mixtures and combinations of any of the foregoing.

15. The method according to claim 2, wherein, in step d) said separating is performed by centrifugation, filtration, sieving, decantation, pressing, plate and frame filtration, vacuum filter belt, basket centrifugation, disk stack centrifugation, and sedimentation.

16. A device for performing the method according to claim 1, said device
comprising
a plurality of hydrolysis vessels, preferably serially arranged, means to add cellulosic biomass, water and enzyme to each hydrolysis vessel, means to add and remove solid fractions and liquid fractions from each hydrolysis vessel,
means to separate a solid fraction from a liquid fraction for each hydrolysis vessel,
means to transfer solid fractions or parts thereof from one hydrolysis vessel to another,
means to transfer liquid fractions or parts thereof from one hydrolysis vessel to another,
means to add enzyme to one or several hydrolysis vessels,
and means to store liquid fractions or parts thereof as final product.
